(19) United States
(12) Patent Application Publication
(10) Pub. No.: US 2022/0095966 A1
(43) Pub. Date: Mar. 31, 2022

(54) METHODS FOR PREDICTING AT LEAST ONE OF THE TOTAL SERUM BILIRUBIN LEVEL AND THE HEMOGLOBIN LEVEL BY USING THE ARTIFICIAL INTELLIGENCE AND THE NON-INVASIVE MEASUREMENT

(71) Applicant: NATIONAL CHENG KUNG UNIVERSITY, TAINAN CITY (TW)

(72) Inventors: Sheng-Hao TSENG, TAINAN CITY (TW); Nan-Yu CHENG, TAINAN CITY (TW); Shih-Yu TZENG, TAINAN CITY (TW); Chun-Yen KUO, TAINAN CITY (TW)

(21) Appl. No.: 17/488,743

(22) Filed: Sep. 29, 2021

Related U.S. Application Data
(60) Provisional application No. 63/085,158, filed on Sep. 30, 2020.

(51) Int. Cl. A61B 5/145; A61B 5/00; A61B 5/1477

(52) U.S. Cl. CPC A61B 5/14503; A61B 5/7264; A61B 5/1477; A61B 5/6823; A61B 5/0075; A61B 5/6825; A61B 5/6822; A61B 5/6828; A61B 5/6829

(57) ABSTRACT

Methods for predicting at least one of the total serum bilirubin level and the hemoglobin level are proposed. The method initially uses the non-invasive measurement to detect one or more sites of the human body for acquiring the corresponding transcutaneous bilirubin and/or hemoglobin level respectively per each site. After that, the artificial intelligence is used to process the acquired results for predicting. Especially, the AI may refer to at least the detected site(s) of the human body(s) and the values of the human body related parameters. Also, the AI may be trained by process a number of measured results and comparing the predicted results with a number of invasive measurement results, such that the correlation coefficient may be approached to 1.0, at least may be about 0.9. Furthermore, neither the used non-invasive measurement nor the used AI is limited.

### CROSS REFERENCE

The non-provisional application claims the benefit of American Provisional Application No. 63/085,158, filed on Sep. 30, 2020, the contents thereof are incorporated by reference herein.

### FIELD OF THE INVENTION

The present invention relates to the methods for predicting at least one of the total serum bilirubin level and the hemoglobin level in the human body, and more particularly to the methods that use the non-invasive measurement to detect one or more sites of the human body and use the artificial intelligence (AI) to process the acquired transcutaneous bilirubin and/or hemoglobin levels respectively for predicting.

### BACKGROUND OF THE INVENTION

Both the bilirubin and the hemoglobin are important for the human medical examination, and usually are expressed as their level (unit: milligrams per deciliter mg/dL). The former is highly related to at least the liver disease, the guts disease and the hemolytic disease, also the latter is highly related to at least the anemia disease and the hyperemia disease. The measurement methods for the human medical examination are essentially grouped into two categories: (1) The invasive blood sampling measurement corresponds to the total serum bilirubin (TSB) and the hemoglobin (Hb); (2) The non-invasive measurement corresponds to the transcutaneous bilirubin (TcB) and the transcutaneous hemoglobin (TcH). Anyway, both categories have their own unavoidable shortcomings. The former usually causes some adverse effects, such as pain, bleeding, and stress, also the measurement results of the latter at different sites of the human body deviate usually from the results of the former.

Further, the existing non-invasive measurement methods generally predict TSB and/or Hb by using the built-in calibration factor(s) or the built-in calibration formula(s) to correct the differences between TcB and TSB and/or the differences between TcH and Hb induced by their physiological differences. In this way, the advantage is that the predicted TSB and/or Hb may be obtained simply and rapidly, but the disadvantage is that both correctness and precision of the predicted TcB and/or the predicted TCH is limited by at least the accuracy and/or the diversities of the available calibration(s). Especially, due to the fixed contents of the built-in calibration, the relation between the invasively measured TcB and/or TcH and the predicted TSB and/or Hb is fixed. In other words, the relation is independent on how the predicted TSB and/or Hb differ from the invasively measured TSB and/or Hb, how many non-invasive measurements are executed, and whether any other message related to the non-invasively measured human body may be referred.

For example, the jaundice occurs in up to 60% of the healthy newborn during the first week of life, especially in Asia, where the hyperbilirubinemia is one of the most common causes for readmission within the first two weeks of life. The neonatal jaundice occurs when a baby has a high level of the bilirubin in the blood. Large amounts of the bilirubin can circulate into the brain tissue and may cause both seizures and brain damages, which is called kernicterus, and it might cause even death in some serve cases. The appearance of the jaundice raises the suspicion of the hyperbilirubinemia, for which the diagnosis should be immediately confirmed and the subsequent treatment will reduce the risk of both morbidity and mortality in the neonates. Although TSB is the current gold standard for diagnosing the neonatal jaundice, however, the venipuncture is uncomfortable for the neonatal and is not suitable for repeated execution in a short period of time. According to the guidelines of the hyperbilirubinemia management in the newborns published by the American Academy of Pediatrics (AAP), the infants should be detected for the development of the jaundice every 8 to 12 hours. For infants who are receiving the phototherapy or whose TSB is rising rapidly, the TSB evaluation should be repeated every 4 to 24 hours. For the readmitted infants, if the TSB level is about 25 mg/dL, the TSB measurement should be repeated every two to three hours. Therefore, the neonates who need to be closely flowed-up on the bilirubin concentration, might receive more invasive blood samplings. In addition, the jaundice in the preterm neonates is difficult to be noticed through the physical observation, and it is impractical to detect via the frequent blood tests.

In contrast, the TcB is an easy, safe, and convenient for the feasible alternative of jaundice screening. The typical bilirubinometers achieve rapid TcB determination through the collection and analysis of light reflected by the skin and subcutaneous tissues, and thus no invasive procedure is necessary. Many studies have documented the reliability in this respect. However, several studies have shown the lack of accuracy of TcB of neonates who are premature, or who already received blue-light phototherapy. Another limitation of the current bilirubinometers is related to the distribution of the extravascular and intravascular bilirubin concentration. One hypothesis is that the cephalocaudal progression of jaundice in newborns is a consequence of diminished capillary blood flow in distal sites of the body. The skin perfusion gradient is believed to account for the uneven deposition of jaundice in newborns, despite the fact that the serum bilirubin level is constant throughout the infant's body. Therefore, the measurement results of TcB at different sites deviate from those of TSB and most of the transcutaneous bilirubinometers are limited to measuring on the body sites above the chest.

Accordingly, the new method(s) for predicting the total serum bilirubin level and the hemoglobin level is desired.

### SUMMARY OF THE INVENTION

The proposed invention uses the artificial intelligence to process the non-invasive measured results, but not use the built-in calibrations as what the conventional skills do. The is a main feature of the proposed invention.

The proposed invention has at least the following advantages. First, it is not limited by the finite built-in calibrations, especially it may minimize the risk that the built-in calibrations are usually based on a finite number of comparisons between the non-invasive measured results and the invasive measured results. Second, the used AI may be continuously trained while it is used to process numerous non-invasive measured result(s) for predicting TSB and/or Hb. Third, the AI may predict according to not only the non-invasive measured results but also the other parameter(s) related to the measured human body. The used parameters may include gender, age and weight of the measured human body, even the age and the weight of the mother of a neonate (if the neonate is non-invasively measured). Hence, the AI may be trained to flexibly predict according to more messages related to the measured human body, but not only according to the non-invasive measured results.

The efficiency of the proposed invention may be emphasized by presenting the correlation coefficient between the TcB/TcH predicted by using the proposed invention and TSB/Hb measured by using other methods, such as the invasive blood sampling measurement. As well-known, TcB and TcH are physiological different from TSB and Hb respectively, and then a calibration factor or even a calibration formula is required to obtain TSB and/or Hb from TcB and/or TcH. Clearly, the value of the correlation coefficient therebetween may indicate how the TcB/TcH obtained by the non-invasive measurement is closed to the invasive blood sampling measurement. In general, for example, the correlation coefficient for TSB and TcB usually is almost less than 1.0, even popularly less than 0.9. Anyway, in some completed tests by using the proposed invention, both the correlation coefficients between TcB and TSB and between TcH and Hb may be improved to about 0.9 or even higher by at least adjusting the detected human body site(s) and/or the value(s) of the used human body related parameters. For the proposed invention, the available correlation coefficient value, about 0.9 or even higher, is obviously not lower than the available values of most of the commercial non-invasive bilirubinometers and hemoglobinmeters. Also, the mean absolute error may be used to further emphasize the efficiency of the proposed invention.

Moreover, the proposed invention needs not to particularly limit the details of the used non-invasive measurement. Anyway, a non-invasive measurement device capable of simply and precisely acquiring measured results from the skin tissue is more suitable. One reason is that good qualify measured results are better base of precise TcB and/or TcH, and another reason is that the flexibility to measure different sites of the human body may increase the available human body related parameters of the used AI. Further, it should be emphasized that the non-invasive measurement detects by analyzing the spectrum of the light reflected from and/or passed through the blood. Hence, many components inside may be detected. Thus, both the bilirubin and the hemoglobin are common in the blood, and then may be easily non-invasively measured. Just for example, in addition to the conventional commercial BiliChek system, the diffuse reflectance spectroscopy (DRS) system described in U.S. Pat. No. 9,345,431 is also applicable to the present invention.

Further, the proposed invention needs not to particularly limit the details of the used AI. Anyway, an AI capable of comprehensively processing both the non-invasively measured results and a large number of parameters is more suitable. One reason is that the proposed invention may predict by referring to more than the non-invasive measured results, and another reason is that some completed tests by using the proposed invention indicate that different combinations of different human body related parameters may significantly affect the correlation coefficient of the predicted results. Just for example, the used AI may have a multiple-layered neural network structure because the relation between TcB and TSB, also between TcH and Hb, of the measured human body is usually non-linear.

The proposed invention may be applied in many conditions, no matter for predicting the bilirubin level and the hemoglobin level in the human body. Just for example, the neonates with the hyperbilirubinemia are generally treated with the blue light phototherapy covering most sites of the body. Under such a condition, the accuracy of the existing bilirubinometers has been reported to be compromised. Although the soles are usually not irradiated during the phototherapy and thus could be a good measurement site, the TcB level at sloes is quiet low. Here, by using the proposed invention, such as using a neural network assisted diffuse reflectance spectroscopy method that is capable of accurately detecting the bilirubin level quantification at the neonate soles, some preliminary tests show that TcB values of the neonates with the phototherapy derived from the proposed method have a correlation coefficient of 0.87 to the TSB and the mean absolute error between TcB and TSB is 1.06 mg/dL.

### BRIEF DESCRIPTION OF THE DRAWINGS

Other advantages, objectives and features of the present invention will become apparent from the following description referring to the attached drawings.

FIG. 1A to FIG. 1B schematically illustrates two essential flowcharts of the proposed invention.

FIG. 2A to FIG. 2E schematically illustrates some basic variations of the essential flowcharts of the proposed invention.

FIG. 3A and FIG. 3B schematically presents an example of the used non-invasive measurement device and some demographic characteristics of the study groups according to some completed examples respectively.

FIG. 4A and FIG. 4B schematically illustrates the absorption coefficient and the reduced scattering coefficient of the enrolled neonates for the four measuring sites respectively.

FIG. 5A to FIG. 5B schematically illustrates the relation between the TSB and the TcB determined using the DRS system and the BiliChek respectively.

FIG. 6 schematically presents the correlation coefficient (r), the mean absolute error (MAE)m and the mean standard deviation (SD) for TSB and TcB at the sole with different input parameters using the DRS system.

FIG. 7A to FIG. 7D schematically illustrates the relation between the TcB versus the predicted TSB by using the ANN model training respectively, wherein FIG. 7A is related to the raw TcB data at the sole, FIG. 7B is related to the raw TcB data at the sole, gestational age and birth height, wherein FIG. 7C is related to the raw data at the sole, and wherein FIG. 7D is related to the raw TcB at the sole, gestational age and birth age.

FIG. 8 schematically presents the correlation coefficient (r) for TSB and TcB of neonates received phototherapy using the DRS system and the BiliChek at different body sites.

FIG. 9A to FIG. 9B schematically illustrates the relation between the TcB versus the predicted TSB by using the ANN model training respectively, wherein FIG. 9A is related to the predicted TSB value of neonates who received phototherapy using ANN with TcB value of sole, and wherein FIG. 9B is related to the predicted TSB value of neonates who received phototherapy using ANN with TcB value of sole and gestation age.

FIG. 10A and FIG. 10B schematically present the available best correlation coefficient (r) and corresponding parameter combinations on some examples by using the DRS system or the BiliChek system for the bilirubin and the hemoglobin respectively.

### DETAILED DESCRIPTION OF THE INVENTION

The invention proposes methods for predicting at least one of the total serum bilirubin level and the hemoglobin level by using the artificial intelligence and the non-invasive measurement. In the proposed invention, the total serum bilirubin level and the hemoglobin level may be predicted at the same time, i.e., both may be predicted while one and only one non-invasive measurement is processed. In other words, TcB and TcH may be obtained by using one and only one non-invasive measurement and then both the total serum bilirubin level and the hemoglobin level may be predicted together accordingly. Although, if necessary, the proposed invention may predict only the total serum bilirubin level or only the hemoglobin level through one and only one non-invasive measurement.

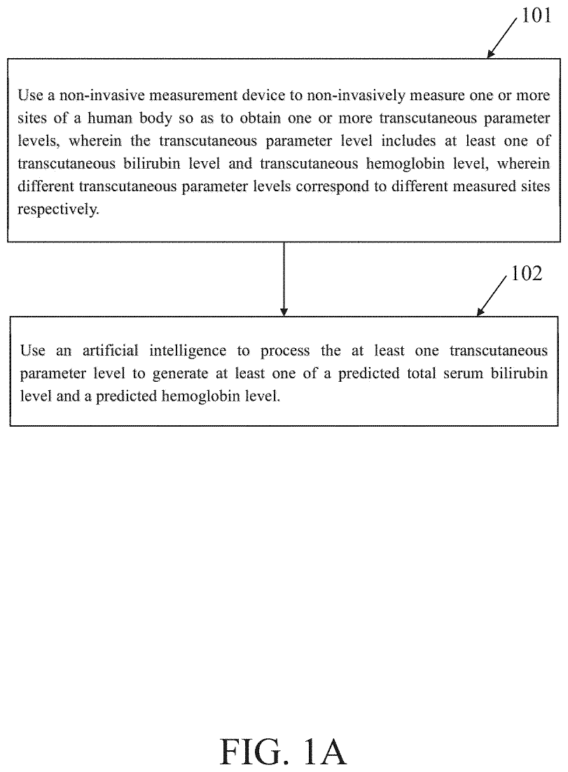

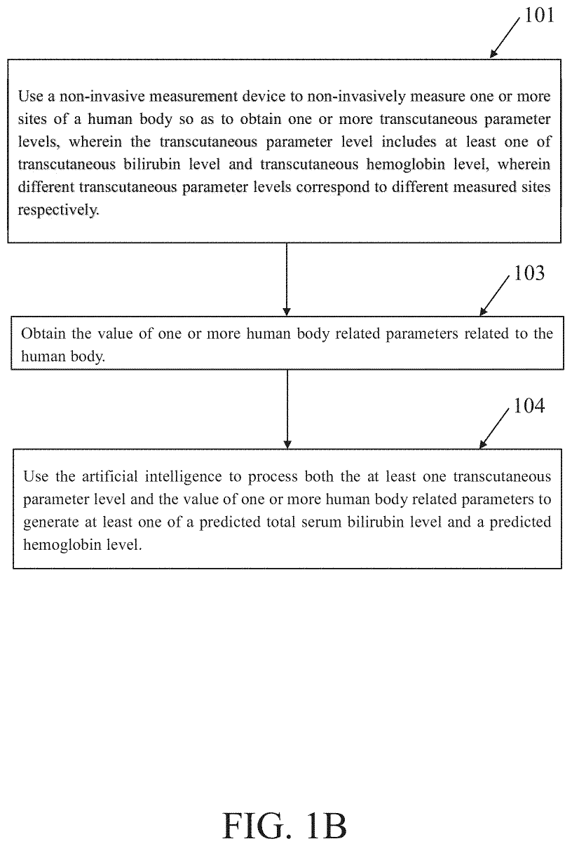

One essential flowchart of the methods proposed by the invention is shown in FIG. 1A. Initially, as shown in block 101, use a non-invasive measurement device to non-invasively measure one or more sites of a human body so as to obtain one or more transcutaneous parameter levels, wherein the transcutaneous parameter level includes at least one of transcutaneous bilirubin level and transcutaneous hemoglobin level, wherein different transcutaneous parameter levels correspond to different measured sites respectively. And then, as shown in block 102, use an artificial intelligence to process the at least one transcutaneous parameter level to generate at least one of a predicted total serum bilirubin level and a predicted hemoglobin level.

Another essential flowchart of the methods proposed by the invention is shown FIG. 1B. Initially, as shown in block 101, use a non-invasive measurement device to non-invasively measure one or more sites of a human body so as to obtain one or more transcutaneous parameter level, wherein each transcutaneous parameter level includes at least one of a transcutaneous bilirubin level and a transcutaneous hemoglobin level, wherein different transcutaneous parameter levels correspond to different measured sites respectively. Next, as shown in block 103, obtain the value of one or more human body related parameters related to the human body. And then, as shown in block 104, use the artificial intelligence to process both the at least one transcutaneous parameter level and the value of one or more human body related parameters to generate at least one of a predicted total serum bilirubin level and a predicted hemoglobin level.

Significantly, by comparing with the conventional non-invasive measurement, one main feature of the proposed invention is the usage of the artificial intelligence. In the proposed invention, each of the total serum bilirubin level and the hemoglobin level is predicated by using the artificial intelligence to process the non-invasive measurement result(s), even to process the human body related parameter(s) together. In contrast, the well-known non-invasive measurement methods use the built-in calibration factor(s)/formula(s) to generate the total serum bilirubin level and/or the hemoglobin level according to the non-invasive measurement result(s). Therefore, the proposed invention has some unique advantages, due to the flexibility, the expandability and the growth of AI. On the one hand, the relation between the non-invasively measured TcB/TcH and the predicted TSB/Hb may be continuously improved by repeatedly comparing the non-invasively measured TcB/TcH with the invasively measured TSB/Hb so as to achieve a better correlation coefficient. In other words, the difference between the predicted TSB/Hb and the invasively measured TSB/Hb may be used to train the used AI, also many non-invasive measurements and invasive measurements may be executed to repeatedly find the difference which many be used to train the used AI. On the other hand, because an AI may generally process more than one parameters at the same time, the invention may process the non-invasively measured TcH/TcB and other parameter(s) together to predict the corresponding TSB/Hb. In other words, the used AI may predict by referring to the non-invasively measured result(s) and the value of one or more human body related parameter, also the used AI may be trained by referring to one or more parameters related to the human body to be non-invasively measured.

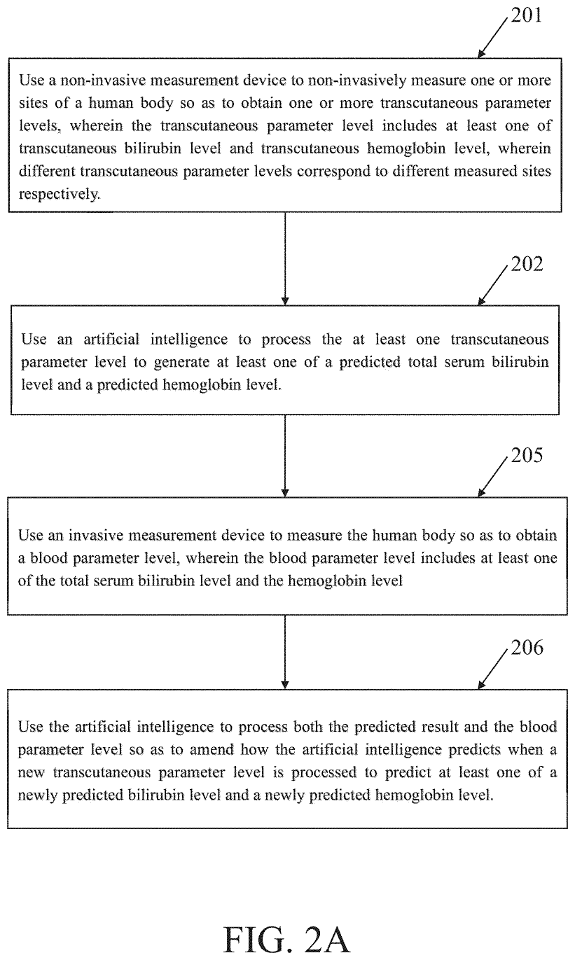

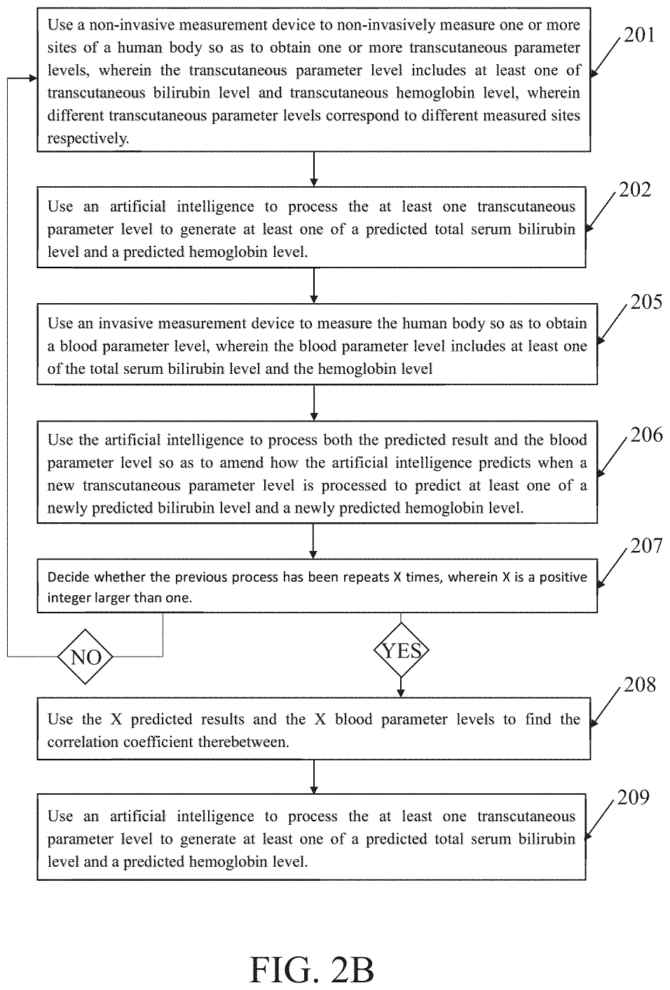

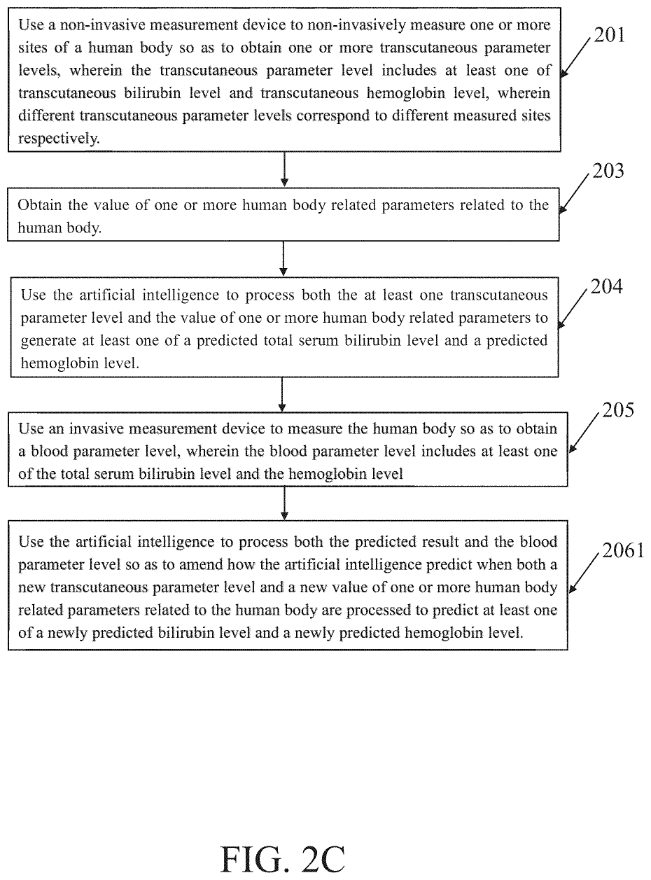

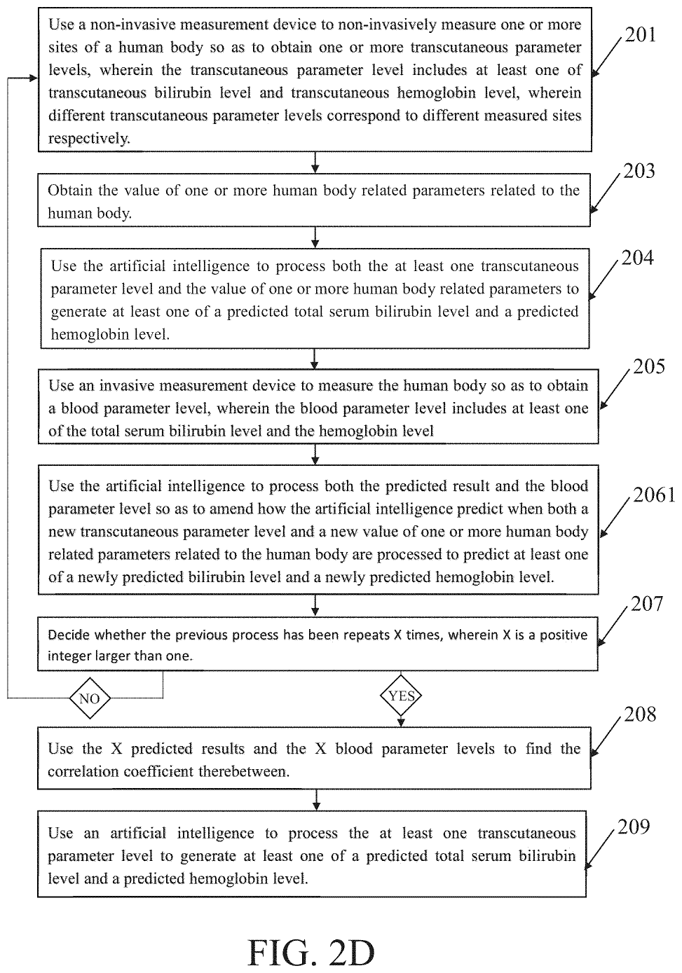

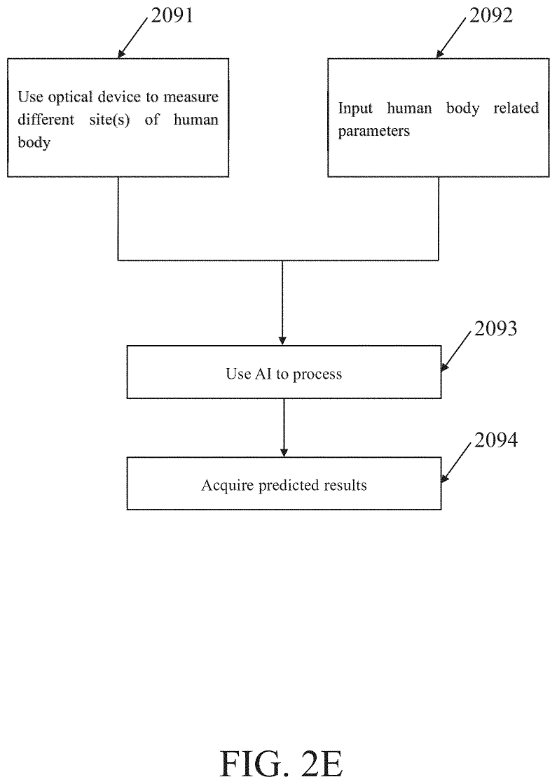

FIG. 2A schematically illustrates a basic variation of the essential flowcharts shown according to the above discussions. The first two steps shown in block 201 and block 202 are equal to that shown in blocks 101 and block 102, and the last two steps shown in block 205 and block 206 are the variations. Here, as shown in block 205, use an invasive measurement device to measure the human body so as to obtain a blood parameter level, wherein the blood parameter level includes at least one of the total serum bilirubin level and the hemoglobin level. Here, as shown in block 206, use the artificial intelligence to process both the predicted result and the blood parameter level so as to amend how the artificial intelligence predicts when a new transcutaneous parameter level is processed to predict at least one of a newly predicted bilirubin level and a newly predicted hemoglobin level. Note that, in different basic variations, both the sequence between block 205 and block 202 and the sequence between block 205 and block 201, may be exchanged, also block 202 and block 206 may be integrated together. Apparently, by comparing the predicted results with the actual measurement result acquired by the invasive measurement device, AI may adjust the way it generates prediction. Therefore, unless AI is perfect enough that there is no room for improvement, the predicted total serum bilirubin level and/or the predicted hemoglobin level generated by an AI is different from the newly predicted total serum bilirubin level and/or the newly predicted hemoglobin level generated by the AI after the AI has adjusted how it generate prediction, even if the new transcutaneous parameter level is equal to the transcutaneous parameter level. In other words, such variations may further enhance both accuracy and correctness of both the predicted total serum bilirubin level and the predicted hemoglobin level.

FIG. 2B schematically illustrates another basic variation of the essential flowcharts according to the above discussions. The first four steps shown in blocks 201/202/205/206 are similar with that shown in FIG. 2A, and the last three steps shown in block 207 to block 209 are the variations. Here, as shown in block 207, decide whether the previous process has been repeats X times, wherein X is a positive integer larger than one. If no, go back to block 201 to repeat the process from block 201 to block 207 again. If yes, go to block 208, use the X predicted results and the X blood parameter levels to find the correlation coefficient therebetween. And then go to block 209, modify the artificial intelligence by referring to the found correlation coefficient. Apparently, the variation shown in FIG. 2B is an advanced version of the variation shown in FIG. 2A, wherein the comparison between the predicted results and the invasive measurement results are repeated X times. In this way, the correlation coefficient between the X prediction results and the X invasive measurement results may be used to adjust how the AI generates its predictions. Because the ideal correlation coefficient is 1.0 which means the prediction results are completely equal to the invasive measurement results, the adjustment of the AI is decided by whether the corresponding correlation coefficient is closer to 1.0. In addition, different one or more sites of the human body may be measured in different times respectively. In this way, the adjustment of the AI is more flexible while more human body related messages may be referred to.

FIG. 2C schematically illustrates another basic variation of the essential flowcharts shown according to the above discussions. FIG. 2C is essentially equal to FIG. 2B except that block 206 is replaced by block 2061, because FIG. 2A corresponds to FIG. 1A but FIG. 2C corresponds to FIG. 1B. In the block 2041, use the artificial intelligence to process both the predicted result and the blood parameter level so as to amend how the artificial intelligence predict when both a new transcutaneous parameter level and a new value of one or more human body related parameters related to the human body are processed to predict at least one of a newly predicted bilirubin level and a newly predicted hemoglobin level. Similar with the above discussion, unless AI is perfect enough so that there is no room for improvement, the newly predicted bilirubin level is different from the predicted total serum bilirubin level and the newly predicted hemoglobin level is different than the predicted hemoglobin level even if the new transcutaneous parameter level is equal to the transcutaneous parameter level and the new value of one or more of human body related parameters is equal to the value of one or more human body related parameters. In other words, both accuracy and correctness of both the predicted total serum bilirubin level and the predicted hemoglobin level are enhanced.

FIG. 2D schematically illustrates one more basic variation of the essential flowcharts shown according to the above discussions. FIG. 2D is essentially equal to FIG. 2B except that block 206 is replaced by block 2061, because FIG. 2B corresponds to FIG. 1A but FIG. 2D corresponds to FIG. 1B. Hence, the repeated descriptions are omitted herein. Anyway, different one or more sites of the human body may be measured in at least two different times respectively, also different values of different one or more human body related parameters may be obtained in at least two different times. In this way, the adjustment of the AI is more flexible while more human body related messages may be referred to.

In addition, FIG. 2E schematically illustrates another basic variation of the essential flowcharts shown according to the above discussions from the perspective of the user. The basic variation is still related to a method for predicting at least one of a total serum bilirubin level and a hemoglobin level. Initially, as shown in block 2091, the user operates an optical device to measure one or more sites of a human body so as to obtain one or more transcutaneous bilirubin levels and/or one or more transcutaneous hemoglobin levels. Then, as shown in block 2092, the user inputs one or more human body related parameters. Finally, as shown in block 2093 and block 2094, the user applies an artificial intelligence to process the optical measurement results and the inputted parameters so as to acquired predication, such as one or more predicted total serum bilirubin level and/or one or more predicted hemoglobin level. Reasonably, for the user of a trained AI, such as the doctor, the nurse or the medical inspector, he/her may directly use the optical device and the trained AI to acquire and process both the human body related parameter(s) and the transcutaneous bilirubin/hemoglobin level(s) one time for generating the predicted total serum bilirubin and/or the predicted hemoglobin level. Reasonably, for the developer of the AI to be used, he/her may repeatedly use the optical device and the AI to acquire and process both the human body related parameter(s) and the transcutaneous bilirubin/hemoglobin level(s) many times so as to gradually adjust the used AI until the correlation coefficient between the predicted result and the invasive measurement result is optimized.

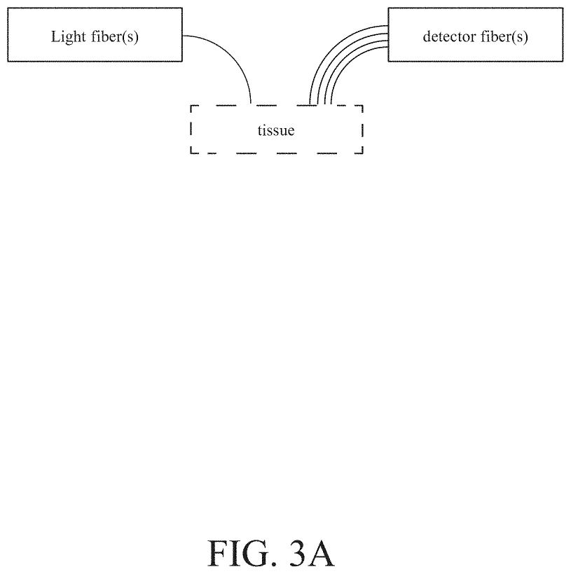

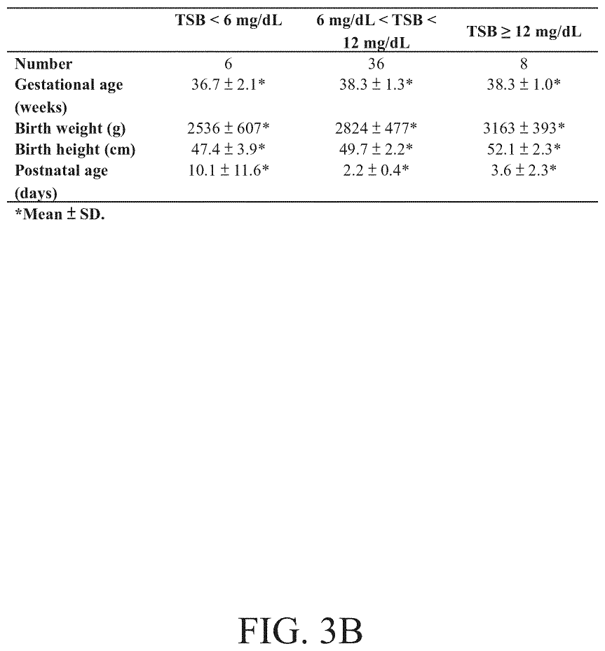

Furthermore, each of these examples discussed above does not have to limit the details of the used AI and the details of the used non-invasive measurement device. Each of them only uses the flexibility, expandability and growth of the AI, also each of them only use the transcutaneous bilirubin and/or hemoglobin level acquired by using the non-invasive measurement device. For example, many of the popular artificial neural networks may be used as the required AI. For example, the used AI may be an artificial neural network with three layers: input layer, hidden layer and output layer, wherein the number of hidden layer size is greater than the single digit to enhance the calculation power. For example, the used AI may be any currently popular software, such as TensorFlow, Theano, Caffe, Torch, MXNet, MATLAB, or other libraries for tensor math. For example, the non-invasive measurement device may be a commercial BiliChek system which may be acquired simply. For example, as shown in FIG. 3A, the non-invasive measurement device may be a multi-fiber probe which is a combination of one light fibers and four detector fibers, wherein the light fiber(s) is used to project light into a tissue to be measured and the detector fiber(s) is used to receive the light reflected from the projected tissue, and wherein different similar examples may be a combination of one or more light fiber(s) and one or more detector fiber(s). For example, the non-invasive measurement device may be diffuse reflectance spectroscopy system presented in U.S. Pat. No. 9,345,431 which may effectively measure a number of sites of the human body. For example, the non-invasive measurement device may be a diffuse reflectance spectroscopy system, wherein a detector fiber is connected to a spectrometer, wherein some other fibers are connected to a xenon flash lamp as a light source through an optical switch, wherein all optical fibers are multimode fibers with a core and a numerical aperture, wherein light passing through the filter us collimated by a lens and coupled to the input port of the multiple fiber switch, and wherein the diffusing probe is equipped with a high scattering Spectralon slab.

Furthermore, each of these examples discussed above does not have to limit the details of the non-invasive measurement, because each only requires the measured transcutaneous bilirubin and/or hemoglobin level. For example, depending on at least the flexibility and the ability of the used non-invasive measurement device, each site of the human body to be measured may be sternum, chest, left sole, right sole, left palm, right palm, forehead, neck, knee, joint, or any distal site of the human body. For example, depending on at least the design of the used AI and the testing results, each of the human body related parameters may be weight, height, age, medical record, health check report, or medication status. For example, depending on at least the design of the used AI and the testing results, each of the human body related parameters may be the birth weight of the human body, the birth height of the human body or the biological parameters related to the mother of the human body, such as gestational age, pregnancy time, and amniotic fluid volume.

More examples and more detailed descriptions of the proposed invention are presented below.

Some completed examples enroll total sixty neonates, wherein fifty are healthy and ten receives phototherapy, and wherein the TSB levels of all neonates ranged from 1.2 mg/dL to 19.9 mg/dL. These neonates are separated into three groups by the TSB value of 6 mg/dL and 12 mg/dL. The demographic characteristics are summarized in FIG. 3B. Moreover, the TSB is determined by using a capillary sample gas analyzer (APEL Neonates BR-200P) and the TcB is determined by using both a Phillip BiliChek and a diffuse reflectance spectroscopy (DRS) system described in U.S. Pat. No. 9,345,431. The TcB measurements are performed three times at each site (such as forehead, sternum, left sole and right sole) and the mean of the three measurements is determined.

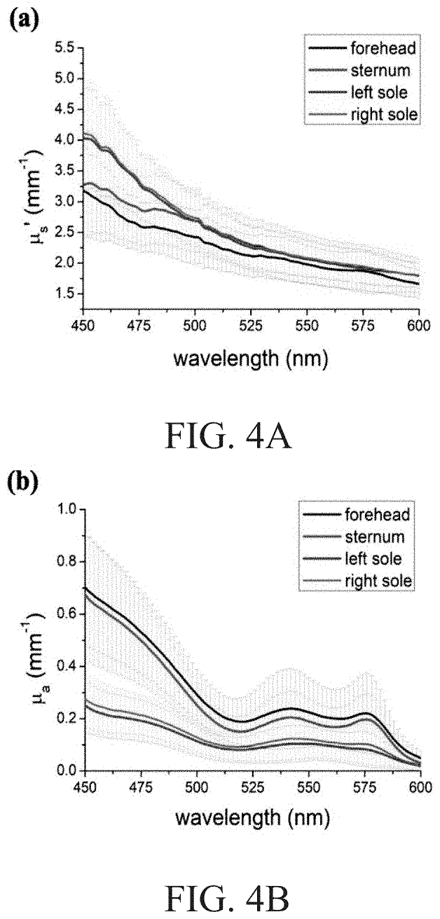

FIG. 4A and FIG. 4B show the average spectra of the subject's skin at the forehead, sternum, left sole and right sole respectively, wherein FIG. 4A presents the absorption coefficients and FIG. 4B presents the reduced scattering coefficients. Within the visible wavelength region (450 nm to 600 nm), four substances are generally considered to dominate the absorption of light in neonates' skin: hemoglobin, oxyhemoglobin, melanin and bilirubin. Notable differences of the absorption coefficients are observed during the forehead, sternum and soles. The cause of these differences is the different blood vessel distribution at the sternum and peripheral limbs. Different skin vascularization, slower blood flow and poorer temperature regulation on the extremities influence the optical property measurements at the different human body sites. Moreover, the optical properties of the lift sole and right sole are slightly different in our study. The difference between the absorption coefficients of right and left soles are 21%, and the differences of scattering coefficients are 4.5% recovered from the two soles. Thus, it is reasonably speculated that the blood circulation of the left and right soles is slightly different, which means they may correspond to different transcutaneous bilirubin and/or hemoglobin level and then to different predicted total serum bilirubin level and/or predicted hemoglobin level.

The raw TcB values recovered by the DRS system is kept to represent the realistic bilirubin concentration of neonatal skin at different sites. Some completed examples indicate that the results of TcB versus TSB at the forehead and sternum. The Person correlation coefficients (r) is 0.87 and 0.89 for TcB and TSB recovered by the DRS system and the BiliChek in all neonates' sternum respectively. The results show that the TcB measured by the DRS system has a slightly lower correlation with TSB and wider measuring range than that measured by the BiliChek at the sternum. In other words, both the DRS system and the BiliChek are workable and useful for the proposed invention. However, the BiliChek shows OOR (out of range) in three neonates whose TSB level are greater than 17 mg/dL, and overestimated TSB by 3.8 mg/dL on average at the sternum. The problem of inaccuracy in high TSB value measured by the BiliChek is mentioned.

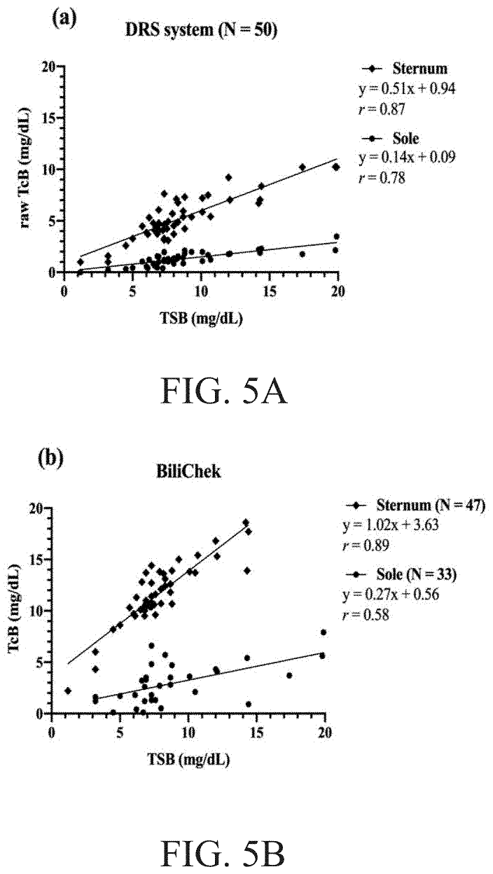

Totally, sixty neonates are enrolled, among them ten received phototherapy before being measured. FIG. 5A and FIG. 5B shows the results of TcB versus TSB at the sternum and the left sole of neonates who does not have a blood-oxygen monitor or other medical devices attached to their soles and never receive phototherapy, respectively. Herein, three sets of data over 17 mg/dl shows OOR for the sternum and 10 sets of data shows OOR and seven sets of data shows zero for the sole. The Pearson correlation coefficient (r) are 0.7 and 0.58 for TSB and TcB recovered by the DRS system and the BiliChek at the left sole, respectively. TcB levels are accompanied by the cephalocaudal progression of jaundice, predicted from the face to the trunk, extremities and finally to the palms and soles. The results support the hypothesis that the cephalocaudal progression of jaundice in newborns is a consequence of diminished capillary blood flow in distal parts of the body. Nearly one third of the TcB measurements recovered by the BiliChek at the soles shows OOR (out of range) or zero values. Although the manual of the BiliChek does not indicate that the measurements could be performed at the neonates' sole, the measurement results at the soles are evidently quite poor. The measurement results obviously do not agree with the BiliChek's claim the measurement range from 0 mg/dL to 20 mg/dl. In contrast, the DRS system has more flexible measurement positions. Thus, for the proposed invention, both the BiliChek and the DRS system may be used flexibly according to the different requirements, such different measured sites of the neonate' body and different TSB levels of measured neonates.

Besides, the measurement of TcB depends mainly on the contribution of extravascular bilirubin concentration rather than that of the intravascular spaces. Thus, it is a physiologically different parameter from TSB. As such, the existing bilirubinometers use the built-in calibration factors to correct the differences between TcB and TSB. In the proposed invention, the AI is applied to predict the values. For example, an artificial neural network (ANN) is used a deep learning tool for predication. Note that the relation between the TSB and the TcB of a site of the human body almost is nonlinear. Therefore, it is beneficial to use a multi-layered neural network structure for modeling this relation. For example, there are three layers in the ANN architecture: input layer, hidden layer and output layer. The TcB values of the neonates' soles and their physiological parameters, such as gestational age, birth wright and birth height are used as input data. In general, the number of hidden layer size is chosen as ten by trial and error. The TSB values are used as output data to train and test for the used ANN model. In addition, both the Pearson's correlation coefficients (r) and the mean absolute error (MAE) are used to evaluate the predicted results.

While an ANN is used and trained to make the prediction of the TSB value, to avoid the imbalanced training and test sets in small size samples, all the TcB data at the sloe recovered by the DRS system are divided into three groups: smaller than 6 mg/dL, 6-12 mg/dL, and greater than 12 mg/dL in TSB. After that, the data in three groups are randomly split into two sets: 70% of the data for training set and 30% of the data for the test set. Eventually, there are 34 data used for training networks and 16 data used for testing the performance in the used ANN model. For example, any currently popular software may be used to implement the required predication, such as TensorFlow, Theano, Caffe, Torch, MXNet, MATLAB, or other libraries for tensor math. The used software is implemented to generate an ANN and the for statement is used to execute a 100 times loop for obtaining the average result. The mean absolute error (MAE) is 1.52 mg/dl, the standard deviation (SD), and the Pearson correlation coefficient (r) is 0.78. The correlation coefficient of the sole through the method of ANN model is similar to that before calibration (r=0.776). Therefore, the method of ANN training does not interfere the original measurement and can be used to obtain the predict scrum bilirubin concentration.

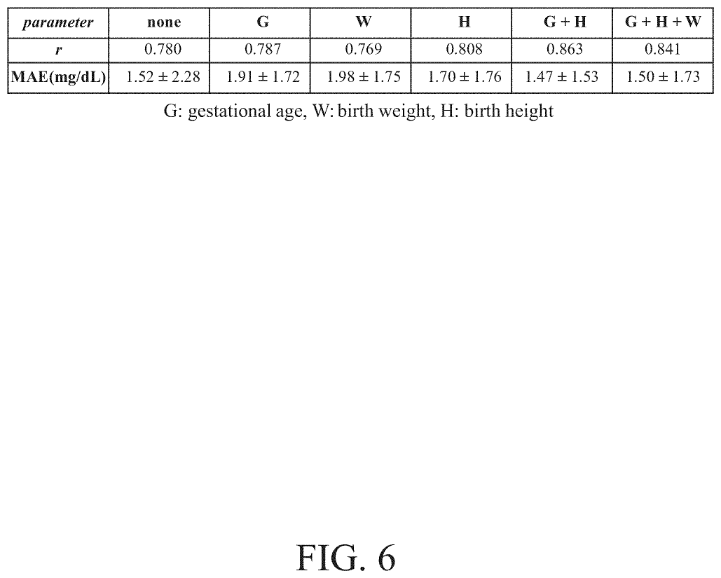

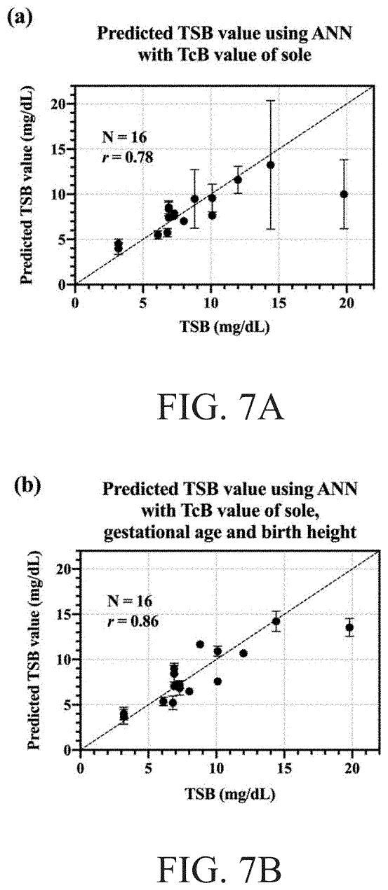

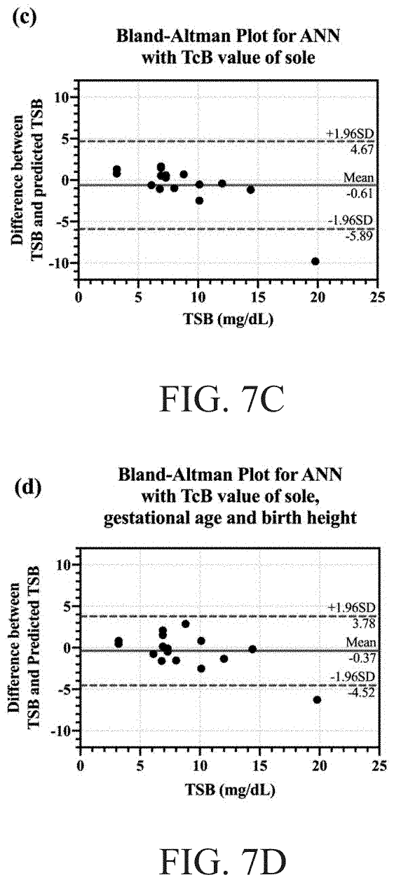

On some completed examples, both the gestational age and the birthweight are important factors to neonatal jaundice. This might be resulted from the skin thickness and newborn maturity with age. The raw mean bilirubin concentration at the soles recovered by the DRS system is about five times smaller than that at the sternum and seven times smaller than the TSB value. Based on the cephalocaudal progression, the gestational age, the birth day, the weight and the height of the newborn are used as input parameters for the ANN mode. The result is shown in FIG. 6. Apparently, a better Pearson correlation (r) result of 0.86 is acquired while using both gestational age and birth height as the input parameters. Moreover, the standard deviation is significantly decreased in the ANN mode as shown in FIG. 7A to FIG. 7D. This result is similar to the BiliChek measurement of sternum (r=0.886) which are the conventional measurement positions. It can be inferred that the sole can also be a good TcB measuring site as well.

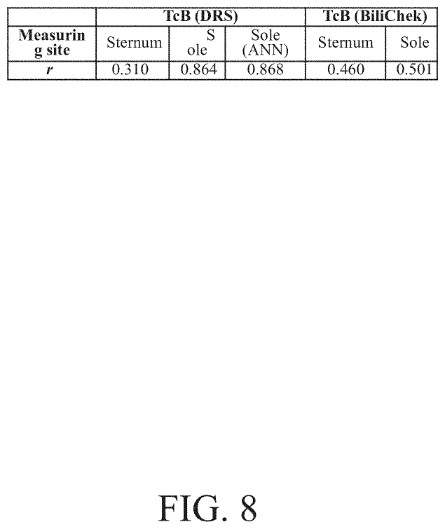

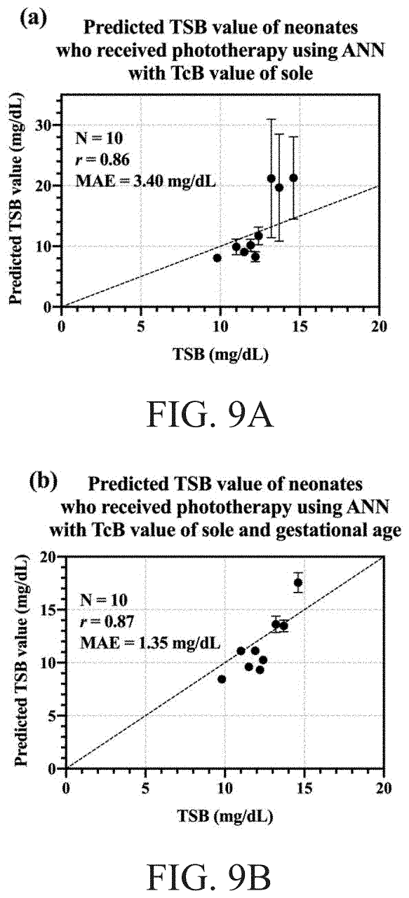

The phototherapy remains an effective therapeutic intervention for neonatal hyperbilirubinemia and it acts on un conjugated bilirubin to a depth of 2 mm from the epidermis. However, the fall in bilirubin level is proportionately greater in the skin than in the serum during phototherapy and the pigmentation occurs as a result of phototherapy, which significantly reduces the correlation between TcB and TSB. It is a clinical difficulty that the current bilirubinometers are not suitable for neonates with jaundice who receive phototherapy. Therefore, it is advantageous to use the soles as the measuring site, as they have relatively lower melanin concentrations and are less influenced by phototherapy. On some completed examples, there are ten neonates who had already received phototherapy, and their TcB data recovered by the DRS and the BiliChek are shown in FIG. 8. The Pearson correlation coefficients are 0.87 and 0.50 for TSB and sole TcB by the DRS and the BiliChek respectively. The BiliChek shows a poorer correlation with TSB and there are one case showing an OOR and another case deviating from the linear fitting line by 100%. On the other hand, in the same measurement, the Pearson correlation coefficient (r) at the sternum is 0.31 by using the DRS and 0.46 vis the BiliChek. Blue light phototherapy converts the bilirubin into water soluble isomers that cab be excreted by the body. Bilirubin in the superficial skin exposed to phototherapy affects TcB more significantly, and this is the reason that the DRS system has a low correlation to TSB values at the sites receiving blue light. However, it is worth noting that measurements taken at the sole had better correlations to TSB than those taken at other sites for both the DRS and the BiliChek systems. The multi-parameter ANN model is also used to predict the TSB value. As shown in FIG. 9A to FIG. 9D, the results of the correlation coefficient (r) do not prominently improve, but both MAE and SD are significantly decreased.

Particularly, the proposed invention also may be applied to predict the hemoglobin level, although these completed examples described above are all processing bilirubin. Note that the non-invasive measurement detects the transcutaneous level of a component in the blood inside the blood vessel by analyzing the spectrum of light reflected from the skin and the tissue. Therefore, after the transcutaneous hemoglobin level is acquired by the non-invasive measurement, the proposed method of using the AI, even referring to one or more related parameters, also may be directly used to predict the hemoglobin level without any significant amendment.

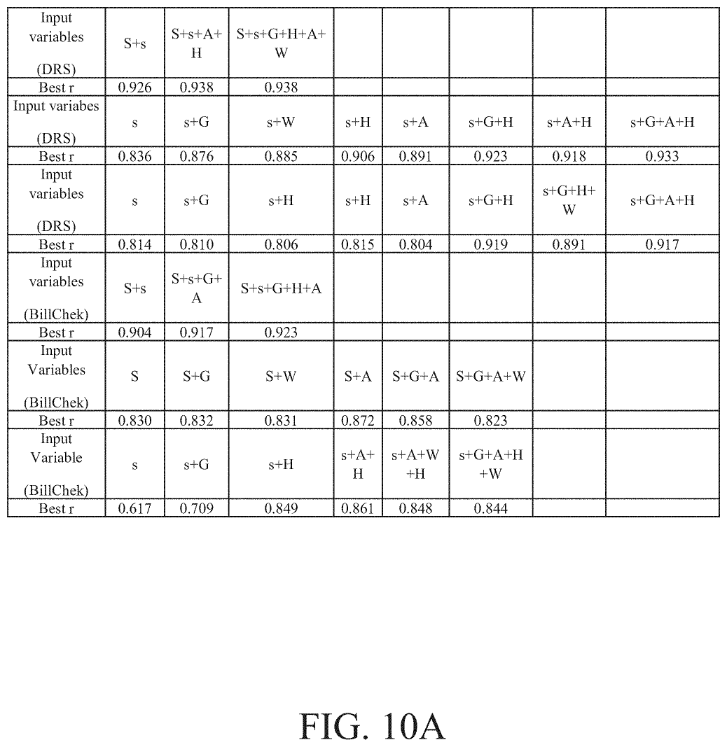

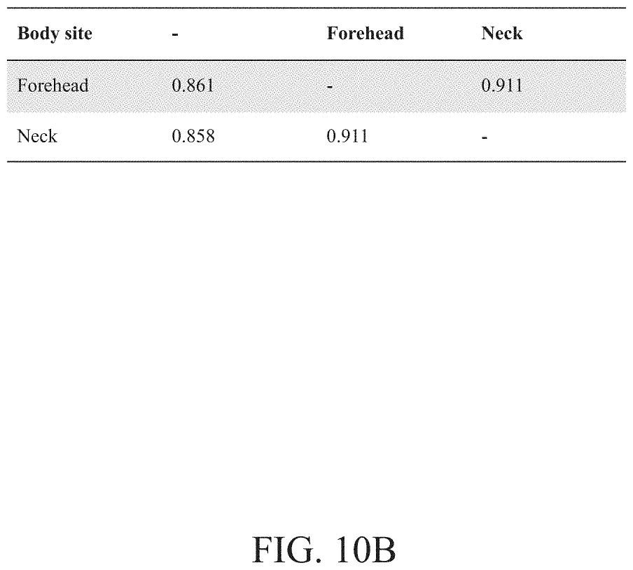

Moreover, some other completed examples are related to the concept of using multiple sites and multiple related parameters to increase the correlation coefficient between TcB and TSB, as shown in FIG. 10A and FIG. 10B. Both the DRS system and the BiliChek system are used respectively, the sites to be measured include sternum (S), sole (s), neck and forehead, and the used parameters include gestation age (G), body height (H), age (A), body weight (W). Also, both the bilirubin and the hemoglobin are measured respectively. Apparently, for the bilirubin, the correlation coefficient value may be improved from 0.617 to 0.923 by using different parameter combinations and the BiliChek system, also the correlation coefficient value may be improved to from 0.804 to 0.938 by using different parameter combinations and the DRS system. Apparently, for the hemoglobin, the correlation coefficient value may be improved from 0.858 to 0.911 by using different parameter combinations. Moreover, it should be emphasized that the correlation coefficient value may be further enhanced by measuring two or more sites of the human body simultaneously. For example, by measuring both neck and forehead, the correlation coefficient value is enhanced to be 0.911 for the hemoglobin. For example, by measuring both sternum and sole, the correlation coefficient value is enhanced to be 0.938 for the bilirubin. Further, it should be emphasized that the optimal parameter combination corresponds to the highest correlation coefficient value is popularly different among different completed examples. In other words, there is no universal optimal parameter combination, but it is necessary to test a variety of possible parameter combinations for each situation to find a certain optimal parameter combination.

As a short summary, the proposed invention uses the artificial intelligent to predict TSB/Hb from TcB/TcH acquired through the non-invasive measurement, wherein the used AI ma further refer to one or more parameters related to the non-invasive measurement and/or the human body to be measured. The usage of AI has some unique advantages. While the conventional skills use a fixed calibration factor/formula to predict TSB/Hb from TcB/TcH, AI provide a flexible approach to predict TSB/Hb from TcB/TcH. Especially, the AI may be continuously optimized when it is used to process a number of invasive measurement results and then predict. Besides, the AI may predict by referring to other parameter(s) but not only the TcB/TcH. Hence, the prediction may be more precise and correct because more messages related to the detected human body are referred. In addition, the proposed invention does not limit the details of the used AI, even the details of the used non-invasive measurement device. Therefore, the proposed invention may be easily implemented, also may be widely applied without being limited by the available hardware and/or software resources.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for predicting at least one of a total serum bilirubin level and a hemoglobin level by using an artificial intelligence and a non-invasive measurement, comprising:
using the non-invasive measurement device to non-invasively measure one or more sites of a human body so as to obtain one or more transcutaneous parameter level, wherein each transcutaneous parameter level includes at least one of a transcutaneous bilirubin level and a transcutaneous hemoglobin level, wherein different transcutaneous parameter levels correspond to different measured sites respectively;
acquiring the value of one or more human body related parameters related to the human body; and
using the artificial intelligence to process both the at least one transcutaneous parameter level and the value of one or more human body related parameters to generate at least one of a predicted total serum bilirubin level and a predicted hemoglobin level;
wherein the one or more human body related parameters comprises at least one of the following: weight, height, age, medical record, health check report, medication status, birth wright of the human body, birth height of the human body, and the biological parameters related to the mother of the human body, such as gestational age, pregnancy time, and amniotic fluid volume.

2. The method according to claim 1, further comprising using an invasive measurement device to measure the human body so as to obtain a blood parameter level, wherein the blood parameter level includes at least one of the total serum bilirubin level and the hemoglobin level.

3. The method according to claim 2, further comprising using the artificial intelligence to process both the predicted result and the blood parameter level so as to amend how the artificial intelligence predict when both a new transcutaneous parameter level and a new value of one or more human body related parameters related to the human body are processed to predict at least one of a newly predicted total serum bilirubin level and a newly predicted hemoglobin level.

4. The method according to claim 3, wherein the newly predicted total serum bilirubin level is different from the predicted total serum bilirubin level and the newly predicted hemoglobin level is different than the predicted hemoglobin level even if the new transcutaneous parameter level is equal to the transcutaneous parameter level and the new value of one or more of human body related parameters is equal to the value of one or more human body related parameters.

5. The method according to claim 2, further comprising:
repeating these steps from using a non-invasive measurement device until using an invasive measurement device X times, wherein X is a positive integer larger than one;
using the X predicted results and the X blood parameter levels to find the correlation coefficient therebetween; and
modifying the artificial intelligence by referring to the found correlation coefficient.

6. The method according to claim 5, wherein different one or more sites of the human body are measured in at least two different times respectively and wherein different values of different one or more human body related parameters are obtained in at least two different times.

7. The method according to claim 1, wherein the one or more sites of the human body to be measured comprise at least one of the following: sternum, chest, left sole, right sole, left palm, right palm, forehead, neck, knee, joint, and any distal site of the human body.

8. The method according to claim 1, further comprising at least one of the following:
the artificial intelligence is an artificial neural network;
the artificial intelligence is an artificial neural network with three layers: input layer, hidden layer and output layer, wherein the number of hidden layer size is greater than the single digit; and
the artificial intelligence is chosen from a group consisting of the following: TensorFlow, Theano, Caffe, Torch, MXNet, MATLAB, other libraries for tensor math, or any combination thereof.

9. The method according to claim 1, further comprising at least one of the following:
the non-invasive measurement device is a commercial BiliChek system;
the non-invasive measurement device is a multi-fiber probe which is a combination of one or more light sources and one or more detector fiber; and
the non-invasive measurement device is a diffuse reflectance spectroscopy system, wherein a detector fiber is connected to a spectrometer, wherein some other fibers are connected to a xenon flash lamp as a light source through an optical switch, wherein all optical fibers are multimode fibers with a core and a numerical aperture, wherein light passing through the filter us collimated by a lens and coupled to the input port of the multiple fiber switch, and wherein the diffusing probe is equipped with a high scattering Spectralon slab.

10. A method for predicting at least one of a total serum bilirubin level and a hemoglobin level, comprising:
processing an optical device to measure one or more sites of a human body so as to obtain one or more transcutaneous bilirubin levels and/or one or more transcutaneous hemoglobin levels;
inputting one or more human body related parameters; and
using the artificial intelligence to process the optical measurement results and the inputted parameters so as to generate one or more predicted total serum bilirubin level and/or one or more predicted hemoglobin level.

11. The method according to claim 10, further comprising at least one of the following:
the one or more sites of the human body to be measured comprise at least one of the following: sternum, chest, left sole, right sole, left palm, right palm, forehead, neck, knee, joint, and any distal site of the human body; and
wherein the one or more human body related parameters comprises at least one of the following: weight, height, age, medical record, health check report, medication status, birth wright of the human body, birth height of the human body, and the biological parameters related to the mother of the human body, such as gestational age, pregnancy time, and amniotic fluid volume.

12. A method for predicting at least one of a total serum bilirubin level and a hemoglobin level by using an artificial intelligence and a non-invasive measurement, comprising:
using the non-invasive measurement device to non-invasively measure one or more sites of a human body so as to obtain one or more transcutaneous parameter level, wherein each transcutaneous parameter level includes at least one of a transcutaneous bilirubin level and a transcutaneous hemoglobin level, wherein different transcutaneous parameter levels correspond to different measured sites respectively; and
using the artificial intelligence to process the one or more transcutaneous parameter levels to generate one or more predicted levels, wherein each predicted level includes at least one of a predicted total serum bilirubin level and a predicted hemoglobin level.

13. The method according to claim 12, further comprising
using an invasive measurement device to measure the human body so as to obtain a blood parameter level, wherein the blood parameter level includes at least one of the total serum bilirubin level and the hemoglobin level.

14. The method according to claim 13, further comprising using the artificial intelligence to process both the predicted result and the blood parameter level so as to amend how the artificial intelligence predicts when a new transcutaneous parameter level is processed to predict at least one of a newly predicted total serum bilirubin level and a newly predicted hemoglobin level.

15. The method according to claim 14, wherein the newly predicted total serum bilirubin level is different from the predicted total serum bilirubin level and the newly predicted hemoglobin level is different than the predicted hemoglobin level even if the new transcutaneous parameter level is equal to the transcutaneous parameter level.

16. The method according to claim 13, further comprising:
repeating these steps from using a non-invasive measurement device until using an invasive measurement device X times, wherein X is a positive integer larger than one;
using the X predicted results and the X blood parameter levels to find the correlation coefficient therebetween; and
modifying the artificial intelligence by referring to the found correlation coefficient.

17. The method according to claim 16, wherein different one or more sites of the human body are measured in different times respectively

18. The method according to claim 12, wherein the one or more sites of the human body to be measured comprise at least one of the following: sternum, chest, left sole, right sole, left palm, right palm, forehead, neck, knee, joint, and any distal site of the human body.

19. The method according to claim 12, further comprising at least one of the following:
the artificial intelligence is an artificial neural network;
the artificial intelligence is an artificial neural network with three layers: input layer, hidden layer and output layer, wherein the number of hidden layer size is greater than the single digit; and
the artificial intelligence is chosen from a group consisting of the following: TensorFlow, Theano, Caffe, Torch, MXNet, MATLAB, other libraries for tensor math, or any combination thereof.

20. The method according to claim 12, further comprising at least one of the following:
the non-invasive measurement device is a commercial BiliChek system;
the non-invasive measurement device is a multi-fiber probe which is a combination of one or more light sources and one or more detector fiber; and
the non-invasive measurement device is a diffuse reflectance spectroscopy system, wherein a detector fiber is connected to a spectrometer, wherein some other fibers are connected to a xenon flash lamp as a light source through an optical switch, wherein all optical fibers are multimode fibers with a core and a numerical aperture, wherein light passing through the filter us collimated by a lens and coupled to the input port of the multiple fiber switch, and wherein the diffusing probe is equipped with a high scattering Spectralon slab.
